(19) United States
(12) Patent Application Publication
(10) Pub. No.: US 2005/0025750 A1
(43) Pub. Date: Feb. 3, 2005

(54) Sperm-induced cellular activation

(76) Inventors: Rafael A. Fissore, Amherst, MA (US); Jason G. Knott, Leicester, MA (US); Manabu Kurokawa, Tokorozawa (JP)

Correspondence Address: Merchant & Gould P.C., P.O. Box 2903, Minneapolis, MN, 55402-0903, US

(21) Appl. No.: 10/747,772

(22) Filed: Dec. 29, 2003

Related U.S. Application Data
(60) Provisional application No. 60/436,474, filed on Dec. 27, 2002.

(51) Int. Cl. A61K 48/00; C12N 5/08

(52) U.S. Cl. 424/93.21; 435/366

(57) ABSTRACT

The present invention provides a method for parthenogenetic activation by injection and subsequent removal of a sperm into a mammalian cell, for example, an oocyte, an embryo, a blastomere, an inner cell mass cell, or a morulae cell.

### CROSS-REFERENCE To RELATED PATENT APPLICATION

This application claims the benefit, pursuant to 35 U.S.C. § 119(e), of provisional U.S. Patent Application Ser. No. 60/436,474, filed Dec. 27, 2002 entitled “SPERM-INDUCED CELLULAR ACTIVATION,” the disclosure of which is hereby incorporated herein in its entirety by reference.

### FIELD OF THE INVENTION

This invention generally relates to artificial reproduction technology. More particularly, the invention relates to methods for parthenogenetic activation of mammalian oocytes, embryos, blastomeres, inner cell mass cells, and morulae.

### BACKGROUND OF THE INVENTION

Artificial reproductive techniques offer numerous benefits in animal husbandry, agriculture, and family planning. For example, the cloning of embryonic cells, together with the ability to transplant the cloned embryonic cells, allows production of genetically identical animals. Cloning by nuclear transfer is preferable to other methods (e.g., embryo splitting or embryonic cell aggregation to produce fetal placental chimeras) because it allows for: (1) the production of genetically identical animals; (2) the selection of specific genetic traits; and (3) the cryogenic storage of embryonic cells until needed.

Fertilization in mammalian species as well as in other animals is characterized by the presence of calcium ion (Ca²⁺) oscillations, which can last for several hours in mammals. See Miyazaki et al. (1986) Dev Biol 118:259-67; Wu et al. (1998) Dev Biol 203:369-81; and Swann & Parrington (1999) J Exp Zool 285:267-75. Such Ca²⁺ oscillations are necessary to trigger egg activation and initiate embryonic development, which consists of a sequence of events including cortical granule exocytosis, resumption of meiosis and extrusion of the second polar body, pronuclear formation, DNA synthesis and first mitotic cleavage. See

### CROSS-REFERENCE TO RELATED PATENT APPLICATION

This application claims the benefit, pursuant to 35 U.S.C. § 119(e), of provisional U.S. Patent Application Serial No. ______, filed Dec. 27, 2002 entitled “SPERM-INDUCED CELLULAR ACTIVATION,” the disclosure of which is hereby incorporated herein in its entirety by reference.

### FIELD OF THE INVENTION

This invention generally relates to artificial reproduction technology. More particularly, the invention relates to methods for parthenogenetic activation of mammalian oocytes, embryos, blastomeres, inner cell mass cells, and morulae.

### BACKGROUND OF THE INVENTION

Artificial reproductive techniques offer numerous benefits in animal husbandry, agriculture, and family planning. For example, the cloning of embryonic cells, together with the ability to transplant the cloned embryonic cells, allows production of genetically identical animals. Cloning by nuclear transfer is preferable to other methods (e.g., embryo splitting or embryonic cell aggregation to produce fetal placental chimeras) because it allows for: (1) the production of genetically identical animals; (2) the selection of specific genetic traits; and (3) the cryogenic storage of embryonic cells until needed.

Fertilization in mammalian species as well as in other animals is characterized by the presence of calcium ion (Ca²⁺) oscillations, which can last for several hours in mammals. See Miyazaki et al. (1986) Dev Biol 118:259-67; Wu et al. (1998) Dev Biol 203:369-81; and Swann & Parrington (1999) J Exp Zool 285:267-75. Such Ca²⁺ oscillations are necessary to trigger egg activation and initiate embryonic development, which consists of a sequence of events including cortical granule exocytosis, resumption of meiosis and extrusion of the second polar body, pronuclear formation, DNA synthesis and first mitotic cleavage. See Kline & Kline (1992) Dev Biol 149:80-9 and Schultz & Kopf (1995) Curr Top Dev Biol 30:21-62.

At least three theories have been proposed to explain the mechanism for Ca²⁺ release. See Swann & Parrington (1999) J Exp Zool 285:267-75. According to a first theory, the sperm may act as a conduit for Ca²⁺ entry into the egg after membrane fusion. A second theory suggests that the sperm may act on plasma membrane receptors to stimulate a phospholipase C (PLC) within the egg to generate inositol 1,4,5-triphosphate (InsP₃ or IP₃). Third, a sperm may induce Ca²⁺ release by a yet unidentified sperm protein. In support of the last theory, sperm cytosolic factors are necessary for oocyte activation (Stice and Robl, 1990; Swann, 1990).

During mammalian fertilization, the sperm induces in the egg an elevation of intracellular calcium concentration ([Ca²⁺]ᵢ) and/or a series of calcium oscillations associated with egg activation. See Kline & Kline (1992) Dev Biol 149 and Schultz & Kopf (1995) Curr Top Dev Biol 30:21-62. Current strategies for the activation of mammalian eggs in the absence of sperm, commonly referred to as parthenogenetic activation, include (1) chemical activation to initially elevate intracellular calcium, followed by down-regulation of Maturation-Propting Factor (MPF); (2) simulation of repetitive fertilization-like calcium rises by treatment with electrical pulses, by injection with sperm extracts, or by incubation in strontium chloride (SrCl₂). See Machaty & Prather (1998) Reprod Fertil Dev 10:599-613 and U.S. Pat. No. 5,496,720.

[Ca²⁺]ᵢ oscillations are a hallmark of mammalian fertilization and several mechanisms have been proposed to explain how they are initiated by the sperm (Schultz and Kopf, 1995; Swann and Parrington, 1999). The most investigated of these hypotheses were the “Conduit hypothesis,” which proposes that the sperm promotes Ca²⁺ influx and, in this manner, makes possible the generation of oscillations (Creton and Jaffee (1995) Dev Growth Differ 37:703-710.), and the “Receptor hypothesis,” which proposes that the sperm acts as a ligand for a receptor in the egg plasma membrane (Schultz and Kopf, 1995). Although the experimental results that emerged from these theories significantly advanced our understanding of the signaling transduction pathways of mammalian fertilization (Miyazaki et al., 1986; Moore et al., (1993) Dev Biol 159:669-678; Williams et al., (1992) Dev Biol 151:288-296), they also exposed severe shortcomings that suggested that these hypotheses were unlikely to explain the action of the sperm (Igusa and Miyazaki, (1983) J Physiol 340:611-632; Mehlmann et al., (1998) Dev Biol 203:221-232.; Williams et al. (1998) Day Biol 198:116-127).

The “Fusion hypothesis,” which proposes that a sperm Ca²⁺ releasing factor(s) is released into the egg after gamete fusion and is responsible for the initiation of the oscillations, is thought to more accurately incorporate the current experimental evidence. First, studies in which gamete fusion was evaluated by dye transfer between gametes have shown that fusion precedes the first [Ca²⁺]i rise by 1 to 5 minutes (Jones et al., (1995). Development 121, 3259-3266; Lawrence et al., (1997) Development 124, 233-241). Second, injections of sperm cytosolic fractions (SF) were able to closely replicate the pattern of oscillations initiated by the sperm (Swann, (1990) Development 110, 1295-1302; Swann and Lai, (1997) Bioessays 19:371-378; Wu et al., (1997) Mol Reprod Dev 46:176-189). Importantly, the activity of SF does not trigger Ca²⁺ responses in eggs from all tested mammalian and non-mammalian species (Homa and Swann, (1994) Hum Reprod 9:2356-2361; Stricker, (1999) Dev Biol 211:157-176; Wu et al., 1997). Furthermore, the advent of intracytoplasmic sperm injection (ICSI), which demonstrated that direct injection of sperm could initiate Ca²⁺ responses comparable to fertilization (Nakano et al., (1997) Mol Hum Reprod 3:1087-1093; Tesarik and Testart, (1994) Biol Reprod 51:385-391), as well as activation and development to term (Kimura and Yanagimachi, (1995) Biol Reprod 52:709-720; Palermo et al., (1992) Lancet 340:17-18), confirmed that interaction of the sperm and egg plasma membranes is not required to initiate fertilization-like oscillations.

Although several candidates have been suggested to be the Ca²⁺ active component of the sperm (Parrington et al., (1996) Nature 379:364-368; Sette et al., (1997) Development 124:2267-2274; Tosti et al., (1993) Mol Reprod Dev 35:52-56), neither its molecular identity, mechanism of release, nor location are presently known. Most of the work on the characterization of the Ca²⁺ active factor has been carried out with “soluble fractions,” so called because they are obtained following sonication or cycles of freeze/thawing of the sperm. Notably, work from Dr. Yanagimachi's laboratory showed that a significant portion of the activation/Ca²⁺ releasing activity is not solubilized by TRITON X-100™ detergent and appears to remain associated with the perinuclear material/theca (Kimura et al., (1998) Biol Reprod 58:1407-1415; Kuretake et al., (1996) Biol Reprod 55:789-795), which may serve as a source of slow release of the factor. Consistent with this evidence, injection of sperm heads previously treated with TRITON X-100™ detergent, which were demembranated but contained perinuclear material, were able to initiate oscillations and activation (Kimura et al., (1998); Perry et al., (2000) Dev Biol 217:386-393), whereas injection of sperm heads treated with trypsin or 1% SDS, which removed the perinuclear material, failed to induce activation (Kimura et al., (1998)). However, the possible deleterious effects of these treatments on the active Ca²⁺ molecule(s) were not tested.

Recent evidence in the literature suggests that at least part of the Ca²⁺ active component(s) may be released away from the fertilizing sperm head. For example, it was shown that cytoplasts from fertilized eggs at the telophase stage were able to trigger activation when fused to metaphase II eggs (Ogonuki et al., (2001) Biol Reprod 65:351-357) In addition, the site from which [Ca²⁺]i rises originate, which are initially observed at the site of sperm penetration, changes, is later observed at the vegetal pole of the egg (Deguchi et al., (2000) Dev. Biol. 218, 299-313; Kline et al., (1999) Dev. Biol. 215, 431-442).

Despite advances in model organisms, methods for parthenogenetic activation have not been readily extended to oocyte activation in large domestic species. In addition, concerns have been raised about the safety of chemical activation agents and the possibility of harm to the resultant cloned animal. These issues acquire greater significance as cloning procedures are used in humans.

Parthenogenesis is the production of embryonic cells, with or without eventual development into an adult, from a female gamete in the absence of any contribution from a male gamete. See U.S. Pat. No. 5,496,720.

Parthenogenetic activation of oocytes can be induced in several ways including: (1) basic treatment with a Ca-ionophore and cytochalasin D combined with cycloheximide; (2) electric impulse; (3) cycloheximide and electric pulse treatments (Bodo et al., (1998) Acta Vet Hung 46:493-500); (4) combined use of calcium ionophores (e.g., A23187) and protein kinase C stimulators (e.g. phorbol esters) (Uranga et al., (1996) Int J Dev Biol 40:51 5-9); (5) oocyte exposure to 7% (v/v) ethanol solution (Lai et al., (1994) Reprod Fertil Dev 6: 771-5); (6) induction using puromycin (De Sutter et al., (1992) J Assist Reprod Genet 9:328-337); (7) incubation of oocytes in strontium ion enriched medium (O'Neill et al., (1991) Mol Reprod Dev 30:2 14-9); and (8) 200 μm thimerosol, which has been observed to induce Ca²⁺ oscillation in pig oocytes (Machaty et al., (1997) Biol Reprod 57:1123-7).

In the field of cellular therapy, methods are also needed for in vitro production of differentiated cell types. Existing approaches involve preparation of an intermediate ES-like cell, which is then induced to differentiate in vitro.

Thus, there exists a need for improved parthenogenetic methods that closely mimic sperm-induced egg activation and for additional methods for in vitro differentiation. To meet these needs, the present invention provides methods for sperm-induced parthenogenetic activation of mammalian cells, including oocyte, embryos, and early embryonic cells.

Therefore, a heretofore-unaddressed need exists in the art to address the aforementioned deficiencies and inadequacies.

### SUMMARY OF THE INVENTION

The present invention provides methods for cellular activation. In one aspect of the invention, a method is provided that includes: (a) introducing a sperm into a mammalian cell, wherein said cell can be an embryo, an oocyte, a blastomeres, an inner cell mass cell, or a morulae cell; (b) culturing the cell for a time sufficient for cell activation; and (c) removing the sperm from the oocyte. Any embryo in need of activation can be treated using the disclosed methods, including a naturally occurring embryo, an embryo fertilized in vitro, a nuclear transfer embryo, or an uniparental embryo.

In various aspects of the present invention, the sperm can comprise an intact sperm or a sperm head. Any sperm that is sufficient for cell activation can be used, preferably a mammalian sperm.

A mammalian cell to be activated and a mammalian sperm can be derived from any mammal, including but not limited to, a human, a primate, a bovine, a caprine, an ovine, a porcine, a feline, a murine, a canine, and a lagomorph (rabbit or hare). In certain aspects of the invention, the sperm is heterologous to the cell to be activated. In other aspects of the invention, the sperm and the cell to be activated are derived from the same species, for example, the methods can employ a human sperm and a human cell to be activated.

The activation methods of the present invention can be combined with one or more conventional methods for cell activation. As a non-limiting example, the methods can further comprise injecting the cell with at least one agent that enhances divalent ionophores, a protein kinase inhibitor, a phosphatase, or a combination thereof. Alternatively or in addition, the cell can be cultured in medium containing Ca²⁺. The method can also include culturing the cell in the presence of factors that promote cellular differentiation.

More specifically, the present invention further provides methods for nuclear transfer cloning. In one aspect of the invention, the method includes: (a) introducing a mammalian donor cell, or a nucleus derived therefrom, into a mammalian enucleated oocyte of the same species as the donor cell or donor cell nucleus, to thereby form a nuclear transfer unit; and (b) activating the oocyte via sperm-induced parthenogenetic activation as disclosed herein. The introduction of a mammalian donor cell can be performed prior to, simultaneous with, or subsequent to activation of the oocyte.

In another aspect of the invention, a method for nuclear transfer cloning includes: (a) activating a mammalian oocyte via sperm-induced parthenogenetic activation as disclosed herein; (b) enucleating the oocyte; and (c) introducing a mammalian donor cell, or a nucleus derived therefrom, wherein the donor cell is of the same species as the oocyte, to thereby form a nuclear transfer unit. The activation of the oocyte can be performed prior to or subsequent to the enucleation of the oocyte.

Also provided are methods to improve the efficiency of in vitro fertilization. In certain aspects of the invention, improved methods for in vitro fertilization include: (a) contacting a mammalian oocyte with a plurality of sperm, whereby the oocyte is fertilized; and (b) activating the oocyte via sperm-induced parthenogenetic activation as disclosed herein. In vitro fertilization can be performed prior to or subsequent to sperm-induced activation. This approach is particularly advantageous when using oocytes from aged individuals.

Activated oocytes of the present invention can be cultured to thereby produced an embryo, for example an embryo comprising from about 1 cell to about 400 cells. Non-human embryos produced by the disclosed methods can be further introduced into a female surrogate and allowed to develop to term. Thus, the present invention also provides activated oocytes, embryos, and cloned non-human mammals that are produced by the disclosed methods.

Accordingly, it is an object of the present invention to provide novel methods for parthenogenetic oocyte activation useful for nuclear transfer cloning and assisted reproduction methods.

### BRIEF DESCRIPTION OF THE DRAWINGS

FIGS. 1A-1B are photographs that depict a Hoescht-labeled egg, which has been fertilized by intracytoplasmic injection of a sperm head. In FIG. 1A, the sperm head (arrow) is observed within the egg. Following sperm enucleation, which was performed as described in the examples, the sperm head (arrow) is observed within the enucleation pipette, as shown in FIG. 1B. The egg metaphase plate and polar body, which also stain with Hoescht 33342 (bright white) remain in the egg following sperm enucleation.

FIGS. 2A-2C depict calcium oscillations in mouse eggs, which were initiated in response to the indicated treatments, and show that in vitro fertilization (IVF) and to intracytoplasmic sperm injection (ICSI) induce similar calcium oscillations, as described in Example 3. Fertilization by IVF (FIG. 2A) and by ICSI using mouse sperm heads (FIG. 2B) initiated oscillations that exhibit similar intervals. ICSI-induced oscillations, which normally cease after 3 to 4 hours (FIG. 2C), were prolonged by culture of the oocyte in the presence of colcemid (FIG. 2D). Calcium oscillations are expressed as the ratio of fluorescence emitted by excitation at 340 nm and fluorescence emitted by excitation at 380 nm (F340/380) as a function of time.

FIGS. 3A-3D depict calcium oscillations in mouse eggs which were initiated in response to the indicated treatments and show that sperm heads, permeabilized sperm, and heterologous sperm can each induce calcium oscillations. Injection of a mouse sperm head (FIG. 3A) or an intact mouse sperm (FIG. 3B) elicited oscillations that were initiated within 30 minutes following injection and that were of similar frequency. Injection of mouse sperm heads that were permeabilized by treatment with TRITON X-100™ detergent (FIG. 3C), accelerated the onset and the frequency of calcium oscillations. Injection of a porcine sperm head induced high frequency calcium oscillations (FIG. 3D). Calcium oscillations are expressed as the ratio of fluorescence emitted by excitation at 340 nm and fluorescence emitted by excitation at 380 nm (F340/380) as a function of time.

FIGS. 4A-4C depict calcium oscillations in mouse eggs, which were initiated in response to the indicated treatments, and show that presence of the sperm head is only transiently required to induce calcium oscillations. As a control, cytoplasm was removed from eggs cultured in the presence of cytochalasin B (added 15 minutes post-ICSI fertilization), which did not affect the pattern of oscillations initiated by the sperm (FIG. 4A). Removal of the fertilizing sperm head 30 minutes post-ICSI fertilization did not result in premature termination of sperm-induced oscillations (FIG. 4B). The sperm head, which was injected into and then removed from a first egg (FIG. 4B), induced calcium oscillations when re-injected into a second egg (FIG. 4C).

FIGS. 5A-L depict the temporal release of sperm factor (SF) following ICSI. [Ca²⁺]i profiles of eggs from which the sperm was removed (A-D) or of new MII eggs injected with recovered sperm heads (E-H) at 15, 30, 60, and 120 min post-ICSI. Control ICSI fertilized eggs were monitored in parallel (I-L). [Ca²⁺]i responses were monitored for 2-3 hours.

FIGS. 6A-C depict the release of SF after IVF. [Ca²⁺]i profiles of a control fertilized egg (A), a spermless egg (B), and of an egg injected with a sperm head recovered approximately 120 min after penetration (C).

FIG. 7 shows the fate of the sperm's perinuclear theca (PT) following fertilization by ICSI. Electron micrographs of mouse sperm 15, 30, 60, and 120 min following ICSI. TEM in the left column are magnified 15,000×. Scale equivalent to 0.7 μm. TEM in right column are magnified 100,000× (B, D, F) and 40,000× (H). Scale equivalent to 0.1 μm (B, D, F) and 0.25 μm (H). (A, B) 15 min after ICSI the PT remains intact (C-F) 30 and 60 min after ICSI, the sperm's PT is exposed and is beginning to become solubilized in the egg cytoplasm (G, H) By 120 min post-fertilization, the PT is completely lost and the sperm chromatin has decondensed. Arrows denote plasma membrane. Arrowheads denote presence and absence of PT. Asterisk denotes electron-dense globules released from the nucleus (Usui, (1996) Mol. Reprod. Dev. 44, 132-140). n; nucleus.

FIGS. 8A-B show that bull sperm retains the PT following injection into mouse eggs. TEM of bull sperm injected into mouse eggs at 30 (A) and 120 min (B) after ICSI. Magnification: 30,000×; inset: 10,000×. Arrows denote PT. Scale equivalent to 0.3 μm.

FIGS. 9A-E depict that bull sperm looses the ability to initiate [Ca²⁺]i oscillations after incubation in mouse eggs. Injection of a fresh bull sperm initiates persistent oscillations in mouse eggs (A). However, removal of the sperm 60 min after ICSI followed by reinjection into a new egg results in near complete loss of the ability to initiate oscillations (B, C). Sperm heads recovered after 120 min post-ICSI are devoid of activity (D, E).

FIGS. 10A-B depict the injection of a male PN is able to initiate [Ca²⁺]i oscillations. [Ca²⁺]i profiles of a MII egg injected with cytoplasm (A), or a male PN (B). The injected PN was aspirated 5-7 h following ICSI.

FIGS. 11A-B show the removal of the sperm's Ca²⁺ releasing ability is not influenced by the stage of the cell cycle. [Ca²⁺]i profiles of mouse eggs injected with mouse sperm incubated for 30 (A), or 120 min (B) in PN stage zygotes.

### DETAILED DESCRIPTION OF THE INVENTION

Various embodiments of the invention are now described in detail. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner in describing the compositions and methods of the invention and how to make and use them. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term. Likewise, the invention is not limited to various embodiments given in this specification.

As used herein, “about” or “approximately” shall generally mean within 20 percent, preferably within 10 percent, and more preferably within 5 percent of a given value or range. Numerical quantities given herein are approximate, meaning that the term “about” or “approximately” can be inferred if not expressly stated.

The present invention provides methods for parthenogenetic activation of a mammalian cell, including oocytes and embryos, via sperm injection and subsequent removal. The disclosed methods also pertain to the development of embryos, or cells derived therefrom (e.g., morulae cells, blastomeres, inner cell mass cells, etc.) and production of differentiated cell types.

Definitions

“Parthenogenetic activation,” as known in the art, refers to development of an ovum or oocyte without fusion of its nucleus with a male nucleus or male cell to form a zygote.

“Oocyte” refers to an unactivated animal egg, i.e. a nucleated or enucleated egg that has not undergone Ca²⁺ oscillations, wherein the Ca²⁺ oscillations resemble those following natural fertilization. Thus, the term “oocyte” encompasses those oocytes that have been subjected to an activation treatment, but which treatment has not elicited Ca²⁺ oscillations resembling those following natural fertilization. A recipient oocyte can comprise a naturally occurring oocyte or an oocyte prepared in vitro, including uniparental oocytes. The term “uniparental oocyte” refers to an oocyte that is derived by gynogenesis or androgenesis such that the oocyte genetic material is derived from a single female or male parent, respectively. “Activated oocyte” refers to an oocyte that resembles an oocyte following natural fertilization. For example, an activated oocyte exhibits Ca²⁺ oscillations, wherein the Ca²⁺ oscillations resemble those following natural fertilization.

“Embryo” refers to the developmental stage that follows implantation and precedes organogenesis. Any embryo in need of activation can be treated using the disclosed methods, including a naturally occurring embryo, an embryo fertilized in vitro, a nuclear transfer embryo, or a uniparental embryo.

The terms “sperm,” “semen,” “sperm sample,” and “semen sample” are used herein interchangeably to refer to the ejaculate from a male animal that contains spermatozoa. The term “sperm head” refers to a sperm subset comprising the nucleus, nuclear material, and/or theca. The terms “theca” and “perinuclear theca” are used herein interchangeably to refer to sperm perinuclear material. “ICSI” refer to intracytoplasmic sperm injection

The term “heterologous,” as used herein to describe a sperm or sperm head, which is used for sperm-induced oocyte activation or for ICSI of an oocyte, refers to a sperm or sperm head that is derived from a species other than the species of the oocyte.

The term “enucleation,” within the context of this application, refers to the removal of a sperm from a recipient egg.

“Sperm-induced parthenogenetic activation”, as disclosed herein, is believed to improve development of embryos produced by nuclear transfer or by artificial fertilization methods. Specifically, the disclosed methods induce persistent calcium oscillations that closely resemble calcium oscillations induced following natural fertilization. Thus, the methods of the present invention may help to overcome abnormalities that result from current methods for manipulation of preimplantation embryos in vitro (e.g., large calf syndrome).

By “medium” or “media” is meant the nutrient solution in which cells and tissues are grown.

“Calcium ionophores” generally refers to agents that allow calcium ions (Ca²⁺) to cross lipid bilayer. Representative calcium ionophores include ionomycin and A23187. Calcium ionophores can be used as described in Liu & Yang (1999) Biol Reprod 61:1-7; Mitalipov et al. (1999) Biol Reprod 60:821-7; Mayes et al. (1995) Biol Reprod 53:270-5; Susko-Parrish et al. (1994) Dev Biol 166:729-39; and U.S. Pat. No. 5,496,720. Typically, oocytes are briefly (e.g., approximately 5 minutes) exposed to the ionophores, optionally in combination with protein kinase inhibitors.

“Protein kinase inhibitor” refers to an agent which inhibits an enzyme that catalyzes the transfer of phosphate from ATP to hydroxyl side chains on proteins causing changes of function of the protein. Representative protein kinase inhibitors that can be used in accordance with the methods disclosed herein are 6-dimethylaminopurine (DMAP), staurosporine, butyrolactone, roscovitine, p34(cdc2) inhibitors, 2-aminopurine and sphingosine.

“Phosphatase” refers to an enzyme that hydrolyzes phosphomonoesters. The preferred phosphatases described herein are phosphatase 2A and 2B.

“Nuclear transfer unit” and “NT unit” refer to the product of fusion between or injection of a donor cell or cell nucleus and an enucleated cytoplast (e.g. an enucleated oocyte). As described further herein below, representative cells that can be used as donor cells or nuclei include embryonic stem (ES) cells, embryonic germ (EG) cells, other embryonic cells such as cells of an inner cell mass. Adult and fetal cells can also be used as donors, including somatic cells that are differentiated and/or proliferating or quiescent.

“Clone” is used herein to described a regenerated organism, wherein all the cells of the organism are genetically identical. Non-human animals can be cloned by nuclear transfer as described herein below. The term “clone” includes cloned embryos, including cloned human embryos, for example as generated for therapies and transplantation.

The present invention provides that brief exposure of sperm to the oocyte cytoplasm is sufficient to initiate oocyte activation that closely mimics the Ca²⁺ oscillations induced following naturally occurring fertilization. As described in Example 1, sperm were labeled with a DNA specific dye, Hoescht 33342, and were injected directly into egg cytoplasm. The injected sperm were allowed to reside in the egg for 15, 30, 60 minutes post-fertilization, after which the sperm were aspirated. Following sperm removal [Ca²⁺] (intracellular calcium) was monitored in the fertilized eggs using Flura-2 fluorescence.

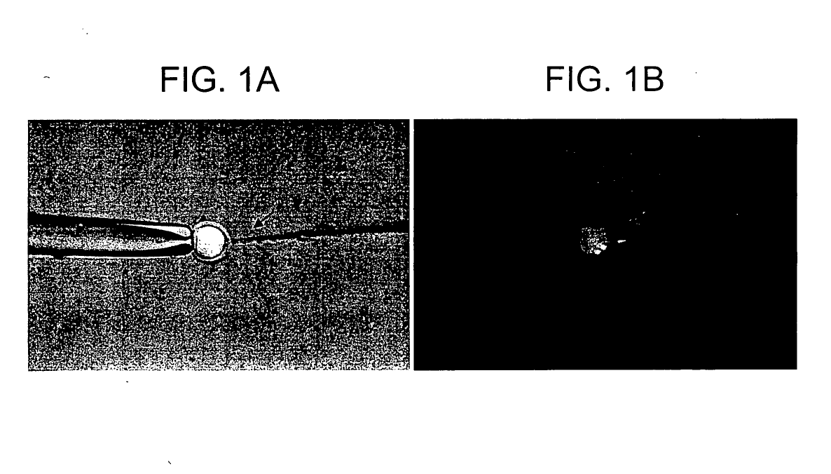

FIG. 1 shows that eggs in which sperm was removed 15 minutes post-fertilization exhibited significantly fewer Ca²⁺ rises and a low percentage of these eggs developed to the two-cell stage. Eggs in which sperm was removed 30 or 60 minutes post-fertilization showed a pattern of Ca²⁺ oscillations that was indistinguishable from naturally fertilized cells. While the inventors do not intend to be bound to a particular mode of operation, the experimental results disclosed herein suggest that an activating molecule may be solubilized away from the sperm in the egg cytoplasm within 30 minutes of entering the egg, after which the sperm nucleus is no longer required to support long term oscillations. The fertilization-like responses of eggs in which sperm was injected and then removed were able to support high rates of parthenogenetic development.

It is envisioned that the disclosed methods for sperm-induced parthenogenetic activation are generally useful in mammalian subjects, including human and non-human subjects, and particularly in those species where ICSI has been shown to work effectively. The term “subject” generally refers to mammalian animals, including livestock animals (e.g. ungulates, such as bovines, buffalo, equines, ovines, porcines, and caprines), primates (e.g. monkeys, chimpanzees, baboons, and gorillas), as well as rodents (e.g. mice, hamsters, rats and guinea pigs), canines, felines, and rabbits. The term “non-human” is meant to include all mammalian animals, especially mammals and including primates other than human primates.

Following a review of the present disclosure, one so skilled in the art of animal husbandry and/or artificial fertilization techniques can readily implement sperm-induced activation methods in other mammalian species. As described further herein, the methods of the present invention can be used for parthenogenetic activation of oocytes produced by nuclear transfer. In addition, the disclosed activation methods are useful in assisted reproduction methods, including in vitro fertilization procedures.

The present invention provides that sperm-induced oocyte activation can variably employ an intact sperm or a sperm head. As noted herein above, a sperm head comprises a sperm nucleus, nuclear material, and/or perinuclear material (i.e., theca). Methods for preparing sperm heads are known in the art. Representative protocols can be found in, but not limited to, Kimura et al. (1998) Biol Reprod 58:1407-15; Kuretake et al. (1996) Biol Reprod 55:789-95; Perreault et al. (1984) Dev Biol 101:160-7; and Uehara & Yanagimachi (1976) Biol Reprod 15:467-70, among other places.

As desired for a particular application, a sperm or sperm head is prepared as a composition further comprising a pharmaceutically acceptable carrier. The term “pharmaceutically acceptable carrier,” generally refers to a pharmaceutically-acceptable material, composition or vehicle, such as a liquid or solid filler, diluent, excipient, solvent or encapsulating material.

The methods for sperm-induced parthenogenetic activation, as disclosed herein, can be used in combination with other activation methods known in the art. Thus, the present invention also provides methods for enhancing oocyte activation. For example, sperm-induced activation methods can further comprise providing one or more calcium ionophores, protein kinase inhibitors, phosphatases or calcium enriched mediums.

Alternatively or in addition to the above-mentioned activation treatments, oocytes can be incubated in a medium enriched for divalent cations. For example, incubation in calcium ion enriched mediums can be carried out as described by Wang et al. (1999) Mol Reprod Dev 53:99-107. Other divalent cations, including magnesium, strontium, and barium, can be utilized in place of calcium. Divalent cation levels can also be increased using electric shock, oocyte treatment with ethanol and treatment of oocytes with caged chelators.

Nuclear Transfer Cloning

In one embodiment of the invention, ES cells and differentiated cells can be used to clone an animal via somatic nuclear transfer. The terms “nuclear transfer” or “nuclear transplantation” refer to a method of cloning, wherein a donor cell nucleus or a donor cell containing a nucleus is transplanted into or fused with a recipient cell (e.g., an oocyte or blastomeres), which is enucleated before or after nuclear transfer. The recipient oocyte is activated, either before, coincident with, or subsequent to nuclear transfer, to thereby initiate embryonic development.

The terms “nuclear transfer unit” and “NT unit” refer to the product of fusion between or injection of a donor cell or cell nucleus and an enucleated cytoplast (e.g. an enucleated oocyte). As described further herein below, representative cells that can be used as donor cells or nuclei include embryonic stem (ES) cells, embryonic germ (EG) cells, other embryonic cells such as cells of an inner cell mass. Adult and fetal cells can also be used as donors, including somatic cells that are differentiated and/or proliferating or quiescent.

The term “clone” is used herein to described a regenerated organism, wherein all the cells of the organism are genetically identical. Non-human animals can be cloned by nuclear transfer as described herein below. The term “clone” includes cloned embryos, including cloned human embryos, for example as generated for therapies and transplantation.

A recipient oocyte can comprise a naturally occurring oocyte or an oocyte prepared in vitro, including uniparental oocytes. The term “uniparental oocyte” refers to an oocyte that is derived by gynogenesis or androgenesis such that the oocyte genetic material is derived from a single female or male parent, respectively. Methods for preparing gynogenetic and androgenetic oocytes are fully described in U.S. patent application Ser. Nos. 09/995,659; 09/697,297; and 60/161,987, which are each incorporated herein in entirety.

The first successful transfer of a nucleus from an adult cell into an enuclated oocyte was reported in 1996 (Campbell et al., (1996) Nature 380:64-66). In most cases, the nuclear transfer (NT) recipient, also called a cytoplast, is derived from a mature metaphase II (MII) oocyte, from which the chromosomes have been removed. However, oocytes in other stages can also be used, for example, oocytes in anaphase and telophase. A donor cell nucleus is placed between the zona and the cytoplast. Fusion is initiated by any suitable technique, such as electrical stimulation. The resultant nuclear transfer unit can be cultured in vitro or in vivo.

Optionally, the nuclear transfer unit, or embryo resulting therefrom, can be implanted into a surrogate female for development and full parturition. By “female surrogate” is meant a female animal into which an embryo of the invention is inserted for gestation. Typically, the female animal is of the same animal species as the embryo, but the female surrogate may also be of a different animal species.

The development of the resultant nuclear transfer unit depends on successful reprogramming of the donor cell nucleus by the cytoplast (Wolf et al., (1999) Biol Reprod 60:199-204). The present invention provides that cytoplast activation can be initiated by sperm-induced parthenogenetic activation, as disclosed herein.

For use as donor cells, embryonic stem cells can be obtained from agriculturally and/or commercially important animals, including chick, cattle, sheep, goats, rabbits, and mink. ES cells can be isolated from any suitable source, including but not limited to the inner cell mass of blastocyst stage embryos, disaggregated morulae, and primordial germ cells. See e.g., PCT International Publication Nos. WOO 95/17500 and WO 95/10599; Canadian Patent No. 2,092,258; Great Britain Patent No. 2,265,909; and U.S. Pat. Nos. 5,453,366; 5,057,420; 4994,384; and 4,664,097. One non-limiting example of a means of producing ES cells or ES-like cells is by culture of inner cell masses of nuclear transfer-derived embryos. Other methods of producing ES or ES-like cells are well-known to those skilled in the art. Representative human ES cells that can be used in accordance with the methods of the present invention include but are not limited to ES cells that give rise to derivatives of three germ layers, for example, those human ES cell lines available from ES Cell International (Melbourne, Australia) and from Wisconsin Alumni Research Foundation (Madison, Wis.). Additional representative human ES cell lines and methods for culturing the same are available from the NIH Human Embryonic Stem Cell Registry, which can be accessed electronically at http://purl.access.gpo.gov/GPO/LPS15792.

Representative isolation and culture methods are known in the art. Preferably, ES cells are maintained as a stable cell line, for example as described in, but not limited to, Joyner (2000) Gene Targeting: A Practical Approach, 2^{nd} ed. Oxford University Press, Oxford, United Kingdom and in Tymms & Kola (2001) Gene Knockout Protocols. Humana Press, Totowa, N.J.; Gerfen et al. (1995) J Neurosci 15:8167-76; Notarianni et al. (1990) J Reprod Fertil Suppl 41:51-6; Notarianni et al. (1991) J Reprod Fertil Suppl 43:255-60; Campbell et al. (1996) Theriogenology 45:287; and in PCT International Publication Nos. WO 01/11019, WO 97/20035, WO 94/26884, WO 94/24274, and WO 90/03432; and in U.S. Pat. Nos. 6,333,192; 6,200,806; and 6,190,910.

In another embodiment of the invention, donor cells used for nuclear transfer comprise cells of an inner cell mass. The term “inner cell mass” refers to a group of cells found in the mammalian blastocyst that give rise to the embryo and are potentially capable of forming all tissues, embryonic and extra-embryonic, except the trophoblast. Representative methods are described by, for example, Keefer et al. (1994) Biol Reprod 50:935-9; Collas & Barnes (1994) Mol Reprod Dev 38:264-7; and Sims & First (1994) Proc Natl Acad Sci USA 91:6143-7.

In yet another embodiment of the invention, donor cells used for nuclear transfer comprise somatic cells. For example, proliferating somatic donor cells are advantageously used in that they are easy to procure and expand in culture and they are amenable to genetic modification. See e.g., Cibelli et al. (1998) Science 280:1256-8, Wilmut et al. (1997) Nature 385:810-3, Kato et al. (1998) Science 282:2095-8, Wells et al. (1999) Biol Reprod 60:996-1005, Baguisi et al. (1999) Nat Biotechnol 17:456-61, Polejaeva et al. (2000) Nature 407:86-90, and Onishi et al. (2000) Science 289:1188-90. See also U.S. Pat. Nos. 6,235,696; 6,215,041; 5,945,577; and 5,843,754, and PCT International Publication Nos. WO 99/05266; WO 99/01164; WO 99/01163; WO 98/39416; WO 98/30683; WO 97/41209; WO 97/07668; WO 97/07669. In a particular embodiment of the invention, nuclei from actively dividing (propagating and proliferating) fetal fibroblasts can be used as nuclear donors according to the procedure described in Cibelli et al. (1998) Science 280: 1256-9.

Donor cells can be genetically modified, for example a heterologous gene (i.e., a marker gene) and/or to express a desired trait. A genetic modification, as used herein, can comprise any alteration of DNA that to a form that is different than its naturally occurring form. Representative gene modifications include nucleotide insertions, deletions, substitutions, and combinations thereof, and can be as small as a single base or as large as tens of thousands of bases. Thus, the term “genetic modification” encompasses inversions of a nucleotide sequence and other chromosomal rearrangements, whereby the position or orientation of DNA comprising a region of a chromosome is altered. A chromosomal rearrangement can comprise an intra-chromosomal rearrangement or an inter-chromosomal rearrangement.

The mechanisms regulating early embryonic development may be conserved among mammalian species, such that, for example, a bovine oocyte cytoplasm can support the introduced, differentiated, donor nucleus regardless of chromosome number, species or age of the donor fibroblast. See e.g., Dominko et al. (1999) Biol Reprod 60:1496-502. Other variations of nuclear transfer cloning methods include the use of young or aged recipient oocytes, in particular oocytes that have extruded a first polar body and that are arrested at metaphase II of meiosis; recloning methods, which involve preparation of a nuclear transfer embryo, which is thereafter used as a donor cell source for additional nuclear transfers; and cryopreservation of nuclear transfer embryos as desired for storage or transport. See e.g., Bondioli et al. (1990) Theriogenology 33:165; Sims & First (1993) Proc Natl Acad Sci USA 90:6143; and U.S. Pat. Nos. 4,994,384; 5,057,420 and 5,453,366.

The development of non-human cloned organisms produced using the methods disclosed herein can be assessed by any suitable technique, including but not limited to external observation, magnetic resonance imaging (MRI), computerized tomography (CT), microscopy, and methods, histological methods, enzymatic assays, biochemical assays, assays to detect changes in gene transcription, including transcription profiling of multiple genes (e.g., chip analysis). General approaches for pathological analysis are described in Porth & Kunert (2002) Pathphysiology: Concepts of Altered Health States, 6^{th} ed. Lippincott Williams & Wilkins, Philadephia, Pennsylvania, and references cited therein. In addition, in vitro phenotypic assays can be performed using cells derived from cloned organisms.

Artificial Reproduction

In another embodiment of the invention, the activation methods disclosed herein are used to facilitate artificial reproduction technologies, including ICSI and in vitro fertilization methods. For example, sperm-induced egg activation may be useful in situations where the male subject has too few mature sperm cells. Similarly, the sperm cells that are incompetent to trigger egg activation, a condition known as globozoospermia. Therefore, for purposes of assisted reproductive techniques, sperm-induced activation can be used to initiate normal embryo development and to preclude the use of artificial activation agents that may have detrimental effects.

In vitro fertilization procedures can be used as described in Long et al. (1993) Mol Reprod Dev 36:23-32 and Trounson A & Gardner D, eds. (1999) Handbook of In Vitro Fertilization, 2^{nd} ed. CRC Press, Boca Raton, Fla. Typically, for example, pooled fresh or cryopreserved semen are processed using the Percoll method as described by Hossain et al. (1996) Arch Androl 37:189-95. Motile sperm are isolated and are provided to a cultured oocyte at a final concentration of 500,000 sperm/ml. Heparin (10 μg/mL; Sigma of St. Louis, Mo.) can be added to the fertilization medium to induce sperm capacitation (Parrish et al., 1988). Eggs are typically incubated with sperm for at least about 4 hours before assaying egg activation.

Monitoring Oocyte Activation

In accordance with the methods of the present invention, a sperm is provided to a recipient oocyte for a time sufficient for oocyte activation. The requisite time may vary when using oocytes of different species. Determination of a time sufficient of oocyte activation in any particular species can be readily accomplished by an initial test of multiple periods of varying duration, for example as described in Example 1. This analysis is simple to perform and should be required only once for optimization of the time required for oocyte activation in a particular species.

Criteria for assessing oocyte activation include, but are not limited to, changes in phosphorylation of cellular proteins, polar body extrusion, and down-regulation of IP₃R, which can be assayed using standard procedures in the art. Representative methods are described herein below. In a preferred embodiment of the invention, oocyte activation is determined by assaying Ca²⁺ oscillations. Preferably, an activation procedure is optimized so that induced Ca²⁺ oscillations most closely resemble Ca²⁺ oscillations following natural fertilization, as described in Example 5.

Kinase Assays

Kinase assays can be used to determine if sperm-induced [Ca²⁺]i oscillations are capable of evoking oocyte activation, and thus determine the efficiency of each of the above combinations of techniques and compositions. Suitable kinase assays include histone H1 and mitogen-activated protein (MAP) kinase assays, which can be performed as described by Fissore et al. (1996) Biol Reprod 55:1261-70. Myelin basic protein (MBP) is assumed to measure mostly MAP kinase activity as shown previously (Fissore et al., 1996). Groups of five eggs are transferred into 5 μL of an H1 kinase buffer solution containing 10 μg/ml aprotinin, 10 μg/ml leupeptin, 10 μg/mL pepstatin A, 500 nM protein kinase A inhibitor, 80 mM β-glycerophosphate, 20 mM EGTA, 15 mM MgCl, and 1 mM dithiothreitol (DTT). See Collas et al. (1993) Mol Reprod Dev 34:224-31. Eggs are lysed by repeated cycles of freezing and thawing and stored at −80° C. until the kinase assay is performed.

Kinase reactions are started by adding 5 μl of a solution containing 2 mg/mL histone H1 (type III-S; available from Sigma of St. Louis, Mo.), 1 mg/ml MBP (Sigma of St. Louis, Mo.), 0.7 mM ATP, and 50 μCi of [ [symbol] ³²P] (Amersham of Arlington Heights, Ill.) to 5 μl of the crude egg lysates. The reaction is carried out for 30 minutes at 30° C., and then terminated by the addition of 5 μl of SDS sample buffer (Laemmli, Nature 227: 680-685 (1970)). Samples are boiled for 3 minutes and loaded onto an about 12% to about 15% SDS-polyacrylamide gel. Control samples typically contain all reaction components, which are combined in the absence of oocytes. Phosphorylation of histone H1 and MBP is visualized by autoradiograph using a CRONEX® intensifying screen (Du Pont De Mours and Company Corporation of Wilmington, Del.) at −70° C., or a similar system.

Polar Body Extrusion and Onset of Cleavage

Oocyte activation can also be determined by visual observation of polar body extrusion, the formation of a pronucleus, and the onset of oocyte cleavage divisions.

Inositol Triphosphate Receptor Levels

Oocyte activation is also readily monitored by assessing levels of inositol triphosphate receptor (IP₃R) which is down-regulated following fertilization, which can be detected at the level of EP₃R RNA transcription and/or protein synthesis. Methods for determining changes in RNA and protein levels are commonly known in the art. Representative protocols can be found in Sambrook & Russell (2001) Cloning: A Laboratory Manual, Cold Spring Harbor Laboratory Press, Cold Srping Harbor, N.Y. and Harlow & Lane (1988) Antibodies: A Laboratory Manual, Cold Spring Harbor Laboratory Press, Cold Spring Harbor, N.Y., among other places.

Calcium Oscillations

In a preferred embodiment of the invention, oocyte activation elicits calcium oscillations, preferably in a pattern and of a duration that closely resembles a pattern and duration of calcium oscillations elicited by natural fertilization. Thus, oocyte activation can also be monitored by assessing [Ca²⁺]i levels, for example as described in Example 5.

Induction of Differentiation

The present invention also pertains to methods for inducing early developmental events and cellular differentiation. As noted herein above, sperm-induced calcium oscillations are implicated in normal development of the embryo after fertilization. Thus, the present invention further provides methods for sperm-induced activation of cells, including blastomeres, inner cell mass cells, and morulae cells, to promote differentiation of such cells to mature cell types, such as cardiocytes, myocytes, neural cells, hematopoietic cells, adipocytes, epithelial cells, endothelial cells, and vascular smooth muscle cells. In vitro preparation of such cell types are useful, for example, in human therapies.

In accordance with this aspect of the invention, a time sufficient for activation comprises a time sufficient for differentiation. A particular cell type or range of cell types produced by the methods disclosed herein can be assessed, for example, by morphological inspection and/or detection of a molecular marker. The term “molecular marker” refers to any measurable molecular quality that is correlated with a cellular identity, including a level of gene expression (e.g., a level of RNA or a level of protein), a protein modification, a protein activity (e.g., an enzyme activity), a level of lipid, production of a lipid type, a lipid modification, a level of carbohydrate, production of a carbohydrate type, a carbohydrate modification, and combinations thereof. Methods for observing, detecting, and quantitating molecular markers are well known to one skilled in the art.

Optionally, the disclosed methods for sperm-induced cellular activation can be combined with existing methods for promoting cellular differentiation. For example, cells employed in the present invention can be further cultured in the presence of growth factors, cytokines, etc. Representative protocols for promoting cellular differentiation and for identifying particular differentiated cell types can be found in for example, but not limited to, Bagutti et al. (1996) Dev Biol 179:184-96; Maltsev et al. (1993) Mech Dev 44:41-50; Maltsev et al. (1994) Circ Res 75:233-44; Miller Hance et al. (1993) Mech Dev 44:41-50.

Without intent to limit the scope of the invention, exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as data are processed, sampled, converted, or the like according to the invention without regard for any particular theory or scheme of action.

### EXAMPLES

### Example 1

### Embryological Methods

Female B6D2F1 mice between 6 and 12 weeks of age were superovulated by sequential injections of 5 IU of ECG (equine gonadotropin from pregnant mare serum; available from Sigma-Aldrich of St. Louis, Mo.) followed by injection of 5 IU of hCG (human chorionic gonadotropin; available from Sigma-Aldrich of St. Louis, Mo.), as previously described (Wu et al., 1998). Metaphase II eggs were collected from the oviducts of stimulated females 14 hours following hCG injection.

Metaphase II eggs were collected in TL-Hepes (Parris et al., 1988; Wu et al., 1998) supplemented with 10% heat-treated fetal calf serum. ICSI was also performed in this medium. Cumulus cells were removed by a brief exposure to hyaluronidase (0.025%). Eggs were cultured in 50 μL drops of KSOM (Specialty Media of Phillisburg, N.J.) under paraffin oil at 36.5° C. in a humidified atmosphere containing 7% CO₂. The zona pellucida was removed by incubating the eggs in acid Tyrode's solution (Hogan et al., 1986).

For IVF experiments, cauda epididymal sperm were capacitated for 1 hour, and 50-100,000 motile sperm/mL were provided to eggs (Hogan et al., 1986). Sperm penetration of eggs was assessed by fluorescent labeling of DNA (e.g., using Hoechest 33342) and observation under epifluoresence within 1 to 2 hours post insemination.

ICSI was performed using Narishige manipulators (W. Nushbaum, Inc. of McHenry, Ill.) under Nikon microscopes essentially as described in Fukami et al. (2001) Science 292:920-3; by Kimura & Yanagimachi (1995) Biol Reprod 52:709-20; and by Wu et al. (1998) Dev Biol 203:369-81. Briefly, sperm were collected and washed in injection buffer (IB; 100 mM KCl and 10 mM HEPES, pH=7.0), and then mixed with an equal volume of 12% polyvinyl pyrrolidone (PVP).

As desired to visualize the sperm during and following ICSI, the sperm's DNA was labeled by incubation with 20 μg/mL Hoechst 33342 (bisbenzimide H 33342; available from Aventis of Strasbourg, France) for 30 minutes at room temperature. Sperm were placed in a 5 μL drop from which a single sperm was aspirated tail first into a 10 μm blunt-ended pipette driven by a Piezo electric unit (Burleigh of Rochester, N.Y.). Several Piezo pulses were applied to separate the sperm head from the tail, after which the sperm head was delivered into the egg by further application of Piezo pulses, which facilitated penetration of the zona pellucida and plasma membrane.

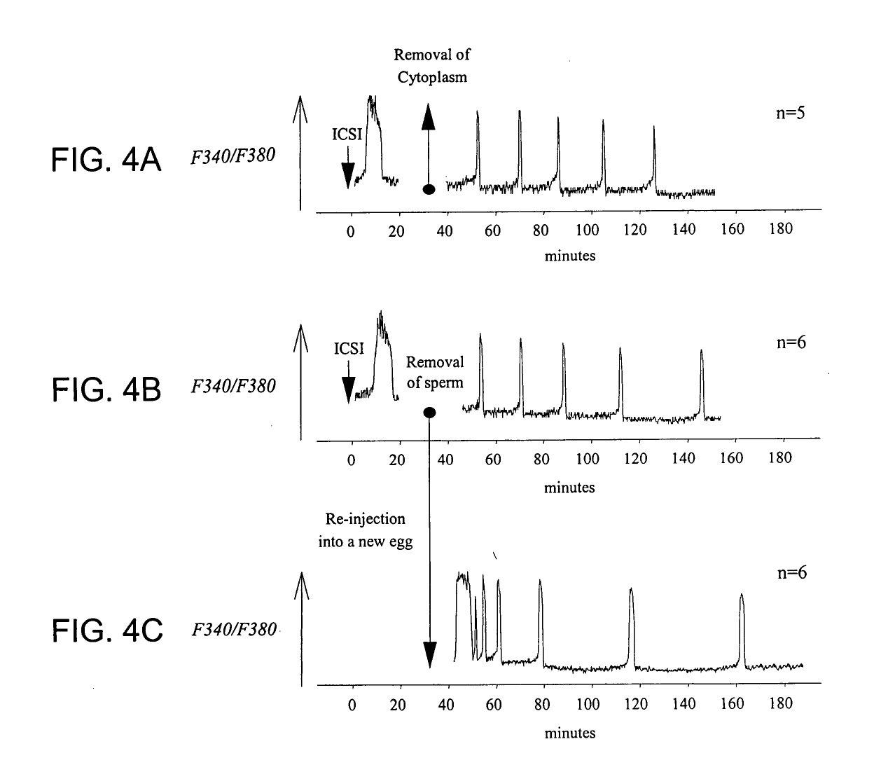

Sperm removal was carried out using the same pipette and Piezo-driven unit. Prior to sperm removal, eggs were placed in a solution of 5 μg/ml cytochalasin B, which optionally also contained 100 ng/ml colcmid, for 15 minutes. See Jones et al. (1995) Development 121:3259-66 and Kono et al. (1995) Development 121:1123-8. To remove a sperm from an egg, a pipette was brought near the Hoechst-stained sperm head, which was identified by brief pulses of UV light, and it was aspirated using an IM-55-2 Narishige syringe. FIGS. 4A and 4B depict Hoescht-labeled sperm following injection into an egg (FIG. 4A) and following its removal from the egg (FIG. 4B).

The enucleated sperm head and surrounding cytoplasm was then brought out of the enucleating drop and several Piezo pulses were applied to remove the surrounding cytoplasm. Following thorough washing, re-injection of the removed sperm was carried out as just described.

### Example 2

### Monitoring Calcium Oscillations

Oocyte activation was monitored by assaying calcium oscillations following a presumptive or candidate activating event, for example by using a calcium indicator such as fura-2 dextran (10 kDa Fura-2D; available from Molecular Probes, Inc. of Eugene, Oreg.) as previously described (Wu and Zhang, 1998). UV illumination was provided by a 75 watt xeon arc lamp, using 340 and 380 nm excitation wavelengths. The intensity of the UV light was attenuated 32-fold with neutral density filters, and a photomultiplier tube was used to quantify the emitted light after passing through a 500 nm barrier filter. The fluorescence signal was averaged for the whole egg. A modified PHOSCAN® 3.0 software program (Nikon, Inc. of New York, N.Y.), which was run on a 486 IBM-compatible system, controlled the rotation of the filter wheel and shutter apparatus to alternate wavelengths. Free [Ca²⁺], was determined from the 340 nm/380 nm ratio of fluorescence. Rₘᵢₙ and Rₘₐₓ were calculated using 10 μM fura-2D in Ca²⁺-free DPBS supplemented with 2 mM EDTA (Rₘᵢₙ) or 2 mM CaCl2 (Rₘₐₓ) and with 60% sucrose to correct for intracellular viscosity. See Grynkiewicz et al. (1985) J Biol Chem 260:3440-50 and Poenie (1990) Cell Calcium 11:85-91. The same solutions were also used to assess background fluorescence. Ca²⁺ measurements are presented as fluorescence ratios of the 340 nm/380 nm excitation wavelengths.

Flura-2 fluorescence was measured in individual eggs placed in 35 μL drops of TL-Hepes medium on a glass coverslip, which was placed on the bottom of a plastic culture dish under paraffin oil. Fluorescence ratios were typically measured every 6 seconds, and readings were taken for 1 second at each wavelength. Oocytes were first monitored for 10-120 seconds to establish baseline [Ca²⁺]ᵢ values, after which recordings were stopped for 2-6 minutes to allow for microinjection or addition of reagents. Recordings were then restarted and continued for 10-30 minutes.

Ca²⁺ responses were compared using either One-Way Analysis of Variance (ANOVA) or Student's t test, according to the number of treatments being compared. Statistical comparisons were performed using the JMP IN software program (SAS Institute Inc. of Cary, N.C.). Differences in Ca²⁺ responses were deemed to be significant if P<0.05.

### Example 3

### ICSI-Induced [Ca²⁺]i Oscillations Are Similar to Those Observed After IVF and Are Prolonged by Colcemid Treatment

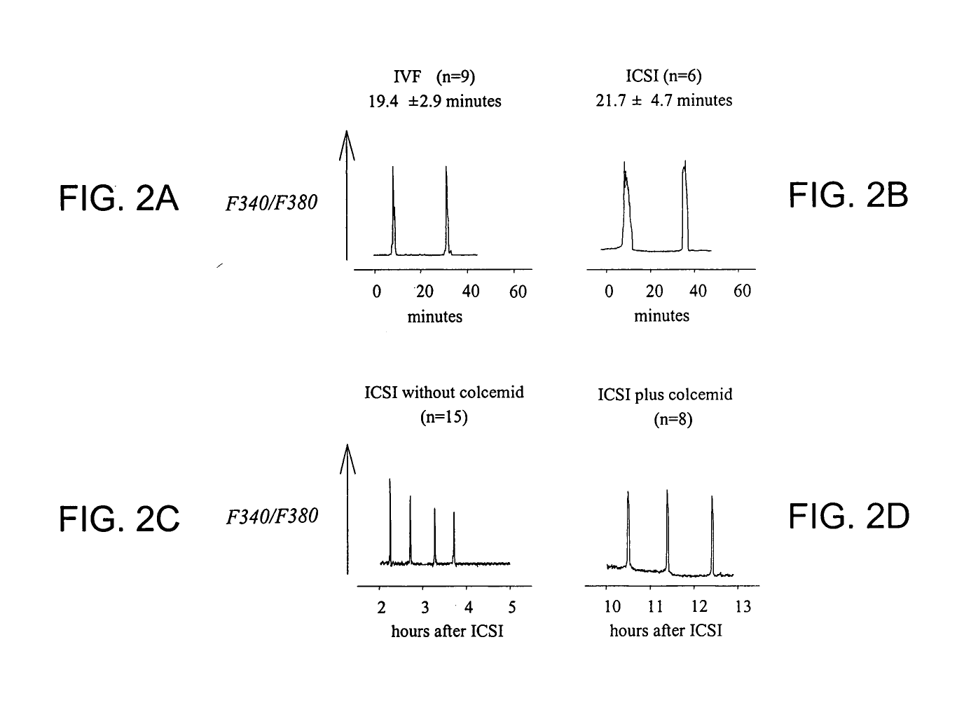

As shown in FIG. 2, fertilization by ICSI faithfully replicates the pattern of calcium oscillations initiated by IVF in mouse eggs, similar to that described by Sato et al. (1999) Cell Calcium 26:49-58 and by Nakano et al. (1997) Mol Hum Reprod 3:1087-93. ICSI performed in the presence of colcemid, a microtubule inhibitor drug that arrests eggs in a metaphase-like stage, significantly prolonged calcium responses. In particular, calcium oscillations persisted for at least about 8-10 hours, as previously reported for IVF-induced oscillations (Jones et al., 1995). These results show that the immediate release, and potentially the persistent release, of the sperm's Ca²⁺ active factor occurs similarly after ICSI and IVF. Thus, ICSI is a valid model to study the release of the sperm's Ca²⁺ active factor during mouse fertilization.

### Example 4

### Separated Sperm Heads or Whole Intact Sperm Similarly Initiate Ca²⁺ Responses

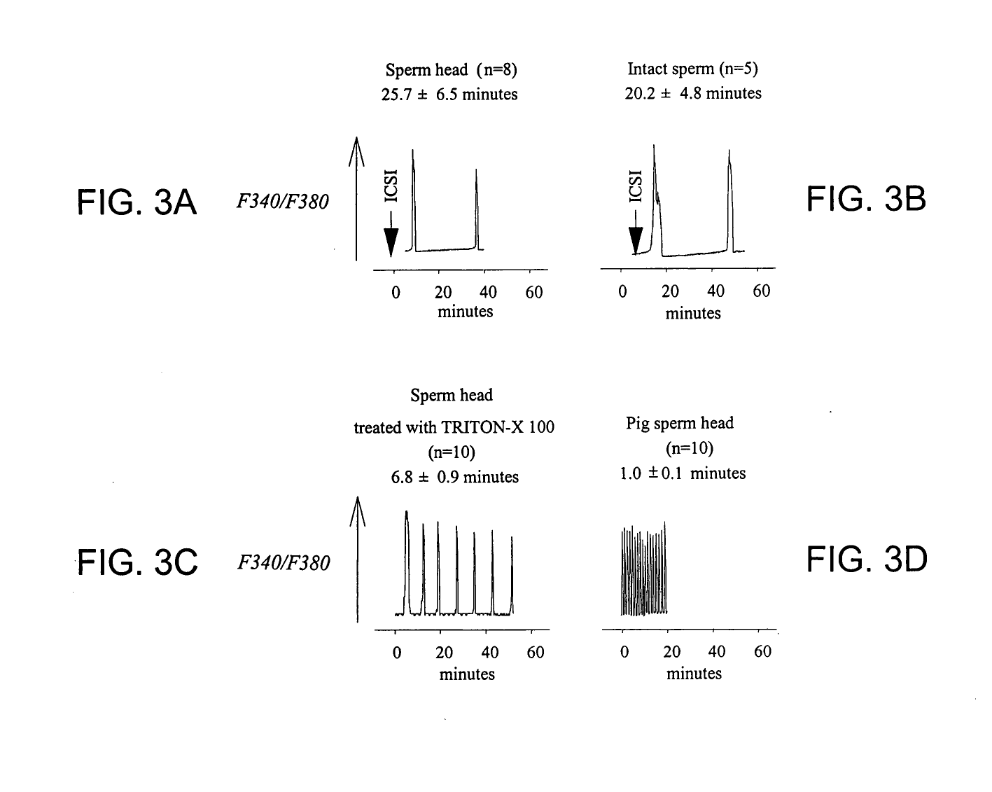

Treatment of sperm prior to injection can affect release of the sperm's Ca²⁺ activity. In particular, injection of intact sperm can delay the onset of calcium oscillations in the egg. See Yanagida et al. (2001) Hum Reprod 16:148-152 and Tesarik & Testart (1994) Biol Reprod 51:385-91. In mouse, both sperm head and intact sperm-initiated calcium oscillations, and a limited delay was observed when using intact sperm. See FIGS. 3A and 3B. As shown in FIG. 3C, treatment of the sperm head with TRITON X-100™ detergent resulted in premature onset of calcium oscillations, which also occurred at an increased frequency. High frequency calcium oscillations were observed in mouse eggs following injection of porcine sperm head, which has significantly greater Ca²⁺ activity than a single mouse sperm, as shown in FIG. 3D.

### Example 5

### Removal of Sperm Does Not Alter Sperm-Induced Calcium Oscillations

A significant proportion of the sperm's Ca²⁺ activity was not solubilized aftertreatment with TRITON X-100™ detergent (Kimura and Yanagimachi, 1995; Perry et al., 2000; Perry et al., 1999), which suggested that this fraction, following fertilization, may remain anchored to the sperm head. In particular, it has been suggested that slow release of the activity from the sperm head could support the long duration of oscillations in mammalian eggs. As disclosed herein, it was discovered that sperm is only transiently required to initiate calcium oscillations. In mouse, residence of an injected sperm in the egg for 15 minutes was sufficient to elicit calcium oscillations. Residence of the sperm in the egg for 30 minutes resulted in oscillations that were indistinguishable from those occurring following persistent presence of the sperm. See Table 1 and FIGS. 4A-4B.

TABLE 1
In Vitro Development of Mouse Eggs Enucleated at 30 Minutes Post-
Fertilization

| | Number of | Pronuclear | 2-Cell | |
|---|---|---|---|---|
| Treatment | Eggs | Formation | Cleavage | Blastocysts |
| Enucle- | 18 | 94% (n = 17) | 94% (n = 17) | 61% (n = 11) |
| ated | | | | |
| Control | 16 | 100% (n = 16) | 100% (n = 16) | 63% (n = 10) |

### Example 6

### Re-Injection of Sperm Into a Second Egg Can Induce Calcium Oscillations

Although a sperm can fully initiate calcium oscillations after a transient residence in an egg (Example 5), a substantial portion of the Ca²⁺ inducing activity remains associated with the sperm head. As shown in FIG. 4C, a sperm head, which was injected and removed from a first egg, elicited calcium oscillations when injected into a second egg.

### Example 7

### Temporal Dynamics For Sperm Factor Responsible For Initiating [Ca²⁺]i Oscillations

In this study, the time required to initiate oscillations was investigated as was whether the sperm must reside continuously in the egg to sustain the oscillations. Also, it was examined whether the sperm's Ca²⁺-releasing activity is associated with the perinuclear theca (PT), and whether disassembly of the PT is necessary for release/dispersal of sperm factor) SF into the ooplasm. Whether the stage of the cell cycle regulates the release of the factor following sperm entry was also determined.

Animals and gametes: B6D2F1 female mice between 6 and 12 weeks of age were superovulated by sequential injections of 5 IU of eCG (all chemicals from Sigma, St. Louis, Mo., unless otherwise specified) followed by injection of 5 IU of hCG, as previously described (Wu et al., 1998). Mouse sperm were obtained from the cauda epididymis of 7- to 11-week-old B6D2F1 males and collected into injection buffer (IB; 75 mM KCl and 20 mM Hepes, pH 7.0). Frozen bull semen (kindly donated by Genex, Ithaca, N.Y.) was prepared according to the Percoll method. Separated sperm were washed once in a Tyrode lactate (TL)-Hepes buffer solution (Parrish et al., 1988) and resuspended in IB. The sperm suspension was then sonicated for 30 s at 4° C. (XL2020; Heat Systems, Farmingdale, N.Y.) and sperm heads resuspended 1 to 1 in 12% polyvinyl pyrrolidone (PVP).

Collection and culture media and in vitro fertilization (IVF): Metaphase II (MII) eggs were collected from the oviducts of stimulated females 14 h following the injection of hCG into TL-Hepes supplemented with 10% heat-treated fetal calf serum (FCS; Gibco, Grand Island, N.Y.) (Wu et al., 1998). The cumulus cells were removed by exposure to 0.025% hyaluronidase for 3-5 min followed by washing in TL-Hepes. Eggs and zygotes were cultured in 50-μl drops of potassium simplex optimized medium (KSOM; Specialty Media, Phillisburg, N.J.) under paraffin oil at 36.5° C. in a humidified atmosphere containing 7% CO2. For IVF, cauda epididymal sperm were capacitated by using human tubal fluid (HTF) medium for 1 h and used at a concentration of 2-3×10⁵ motile sperm/ml (Quin et al., 1985).

ICSI, enucleation, and sperm head reinjection: ICSI was carried out as previously described (Kimura and Yanagimachi, 1995; Fukami et al., 2001) using Narishige manipulators under Nikon microscopes. All manipulations were carried out in drops of flushing and holding media (FHM; Specialty Media) under light mineral oil. For ICSI, sperm were washed in IB and mixed 1-1 with 12% PVP. When needed, the sperm's DNA was labeled by incubation with 3 μg/ml Hoechst 33342 for 30 min at room temperature (RT). Sperm were placed in a 5-μl drop from which a single sperm was aspirated tail first into a 10-μm blunt-ended pipette driven by a Piezo electric unit (Burleigh, Rochester, N.Y.). For mouse sperm, several Piezo pulses were applied to separate the head from the tail, after which the sperm head was delivered into the egg by further application of Piezo pulses to penetrate the zona pellucida and plasma membrane. Enucleation (term used here exclusively to indicate removal of the sperm head) was carried out by using the same pipette and Piezo-driven unit. Prior to the procedure, eggs were placed in 5 μg/ml cytochalasin B (Kono et al., 1995) for 10-20 min to facilitate enucleation and increase survival rates. To enucleate, the pipette was brought near the Hoechst-stained sperm head, which was identified by brief pulses of UV light, and the sperm head was aspirated by using an IM-55-2 Narishige syringe. The enucleated sperm head and surrounding cytoplasm were brought out of the enucleating drop, and Piezo pulses were applied to remove the surrounding cytoplasm. Following washing in IB, sperm heads were reinjected into fresh MII eggs. In experiments in which the sperm was removed following IVF, the fertilized eggs were exposed to 1 μg/ml Hoechst 33342 for 20 min at RT. Enucleation was as described, although the aspirated sperm contained an intact tail that was separated prior to injection into a new egg.

In experiments in which in vitro development of enucleated eggs was evaluated, the fertilized spermless eggs were kept in the presence of 5 μg/ml cytochalasin B for 4 h to block extrusion of the second polar body and in this manner maintain a 2n chromosomal complement.

[Ca²⁺]i monitoring: [Ca²⁺]i monitoring of Fura-2 acetoxymethylester (AM)-loaded eggs (1 μM; Molecular Probes) supplemented with 0.02% Pluronic Acid at RT for 20 min was carried out as previously described (Gordo et al., 2002). In brief, [Ca²⁺]i values were monitored by using a Nikon microscope fitted for fluorescence measurements. Several eggs were monitored simultaneously by using the software Image 1/FL (Universal Imaging, Downington, Pa.), and images were acquired by using an SIT camera (Dage-MTI, MI City, Ind.) attached to an amplifier (Video Scope International Ltd., Sterling, Va.). Fluorescence ratios were obtained every 20 s, and values were reported as the ratios of 340/380 nm fluorescence.

Electron microscopy: Changes in the sperm's PT were monitored by transmission electron microscopy (TEM) at 15, 30, 60, and 120 min post-ICSI in mouse eggs. TEM was performed as described earlier (Wu et al., 1998; Abbott et al., 2001). In brief, fertilized eggs were fixed with 2% glutaraldehyde and 4% paraformaldehyde in 0.05 M sodium cacodylate buffer (pH 7.2) for 2 h. Fixed eggs were washed in cacodylate buffer and then postfixed with 1% OsO4 and 0.8% potassium ferricyanide for 60 min. Dehydration was carried out by processing eggs through increasing concentrations of ethanol. Eggs were then embedded in epoxy resin and polymerized at 70° C. Eggs were sectioned by using a Reichut-Jung Ultracut E ultramicrotone, and thin sections were double stained with uranyl acetate and lead citrate. Sections were examined under a Philips CM10 transmission electron microscope at an accelerating voltage of 80 kV. For each treatment, at least three eggs were evaluated.

Initiation of [Ca²⁺]i Oscillations and Development in Fertilized Spermless Eggs

To determine whether or not the initiation of fertilization-associated [Ca²⁺]i responses relies on the continuous presence of sperm, 30 min after ICSI, sperm were removed and the [Ca²⁺]i responses and in vitro development of fertilized spermless eggs was examined. Removal of sperm 30 min post-ICSI did not impact the pattern or persistence of [Ca²⁺]i oscillations compared with fertilized unmanipulated controls for 3 h during which oscillations were monitored (data not shown). What is more, fertilized spermless eggs supported preimplantation development, and after 5 days in culture, spermless eggs cleaved (17/18; 94%) and developed to the blastocyst stage (11/17; 61%) with rates comparable to those observed in control zygotes (16/16; 100%, and 10/16; 63%).

Temporal Release of SF During Fertilization

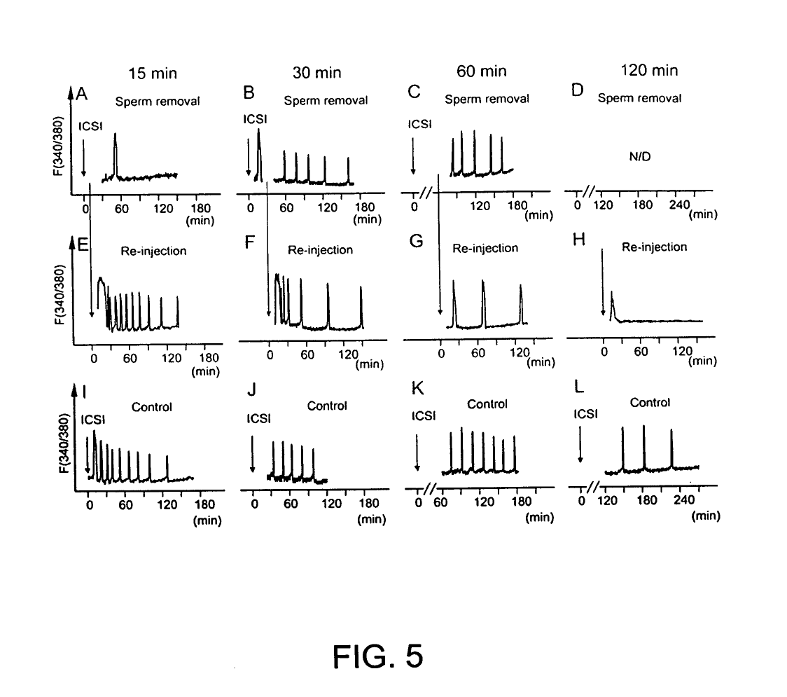

Having established that 30 min after sperm entry was sufficient to trigger fertilization-like [Ca²⁺]i oscillations, it was next investigated the temporal, and possible complete, release of SF during fertilization. Sperm were withdrawn from eggs at 15, 30, and 60 min post-ICSI and the [Ca²⁺]i responses in these spermless eggs monitored. In addition, and almost simultaneously, the recovered sperm heads were reinjected into new MII eggs and the [Ca²⁺]i responses, activation rates, and cleavage to the two-cell stage induced in these eggs were evaluated. Fertilized eggs enucleated 15 min post-ICSI were unable to mount persistent oscillations (FIG. 5A and Table 2; P<0.05) and, consequently, only a small number of these eggs cleaved to the two-cell stage. In contrast, reinjection of sperm heads recovered at this time point exhibited maximal Ca²⁺ activity, as evidenced by the ability to trigger fertilization-like oscillations in new MII eggs (FIG. 5E and Table 2), and induced high rates of cleavage to the two-cell stage (Table 3). As observed for eggs enucleated at 30 min, removal of sperm 60 min after ICSI did not alter the pattern of [Ca²⁺]i oscillations and, as expected, high cleavage rates were observed in these eggs (FIG. 5C and Table 3). Importantly, although reinjection of sperm heads recovered at 30 or 60 min post-ICSI consistently triggered [Ca²⁺]i oscillations, the initiated [Ca²⁺]i responses exhibited progressively longer intervals (FIGS. 5F and G and Table 1; P<0.05) and, as a result, fewer zygotes cleaved to the two-cell stage

TABLE 2
Characterization of [Ca²⁺]_{I} responses in fertilized spermless (enucleated) eggs and in
eggs injected with sperm recovered after residence in mouse eggs

| Time after | | No. eggs | % Eggs with >2 | No. Ca²⁺^{a} rises | |
|---|---|---|---|---|---|
| ICSI | Treatment | examined | rises | for 1st h | Intervals^{b} |
| <15 min | Enucleated | 12 | 33.3 | 0.8 ± 0.3^{c} | 43.6 ± 9.6^{c} |
| | Reinjection | 5 | 100 | 4.6 ± 0.8 | 16.3 ± 2.9 |
| | Control | 7 | 100 | 6.0 ± 0.9 | 12.3 ± 1.9 |
| 30 min | Enucleated | 13 | 92.3 | 3.1 ± 0.4 | 25.5 ± 4.5 |
| | Reinjection | 7 | 100 | 4.9 ± 1.7 | 35.5 ± 9.9^{c} |
| | Control | 10 | 100 | 4.0 ± 0.9 | 21.2 ± 3.5 |
| 60 min | Enucleated | 11 | 100 | 4.5 ± 0.6 | 17.7 ± 2.8 |
| | Reinjection | 6 | 100 | 1.8 ± 0.8^{c} | 45.1 ± 5.9^{c} |
| | Control | 7 | 100 | 4.3 ± 1.7 | 19.2 ± 3.6 |
| 120 min | Reinjection | 8 | 0 | 1.0 ± 0.0 | |
| | Control | 6 | 83.3 | 0.83 ± 0.2 | 54.7 ± 5.2 |

^{a}Mean ± SEM of number of [Ca²⁺]_{I} rises during the first hour of sperm injection.
^{b}Mean ± SEM of intervals between [Ca²⁺]_{I} rises during the first hour post injection; all rises observed during the first 2 h of monitoring were used to calculate this number.
^{c}Values of treatments with a superscript are significant different than controls within column and row (Time after ICSI) (P < 0.05).

It was next examined whether the sperm's Ca²⁺-releasing activity becomes completely dissociated from the sperm head. To accomplish this, sperm were withdrawn from eggs 120 min post-ICSI and reinjected into different MII eggs (FIG. 5H). Reinjection of these sperm failed to trigger [Ca²⁺]i oscillations in all eggs examined (n=8) and, as expected, none of them formed a pronucleus (PN) (Table 3). It is worth noting that injection of comparable volumes of cytoplasm from these eggs, which presumably contain all the sperm's SF, failed to initiate oscillations (data not shown), demonstrating that, although the factor(s) is completely released, it is greatly diluted in the ooplasm.

TABLE 3
Activation and cleavage to the two-cell stage of fertilized spermless
(enucleated) eggs and eggs injected with recovered sperm heads

| | | | % Eggs | |
|---|---|---|---|---|
| | | No. eggs | Developed to | |
| Time after ICSI | Treatment | examined | PN | 2 Cell |
| <15 min | Enucleated | 12 | 58.3 | 33.3 |
| | Reinjection | 5 | 100 | 80.0 |
| | Control | 7 | 100 | 100 |
| 30 min | Enucleated | 13 | 100 | 92.3 |
| | Reinjection | 7 | 100 | 86.0 |
| | Control | 10 | 100 | 100 |
| 60 min | Enucleated | 11 | 100 | 91.0 |
| | Reinjection | 6 | 100 | 33.3 |
| | Control | 7 | 100 | 86.0 |
| 120 min | Reinjection | 8 | 0 | 0 |
| | Control | 6 | 100 | 100 |

Note:
Activation and development were monitored in the same eggs subjected to [Ca²⁺]_{I} monitoring in Table 2. Eggs were considered activated when they had one or two PNs 5-7 h post-ICSI. Cleavage to the two-cell was evaluated 15-18 h post-ICSI.

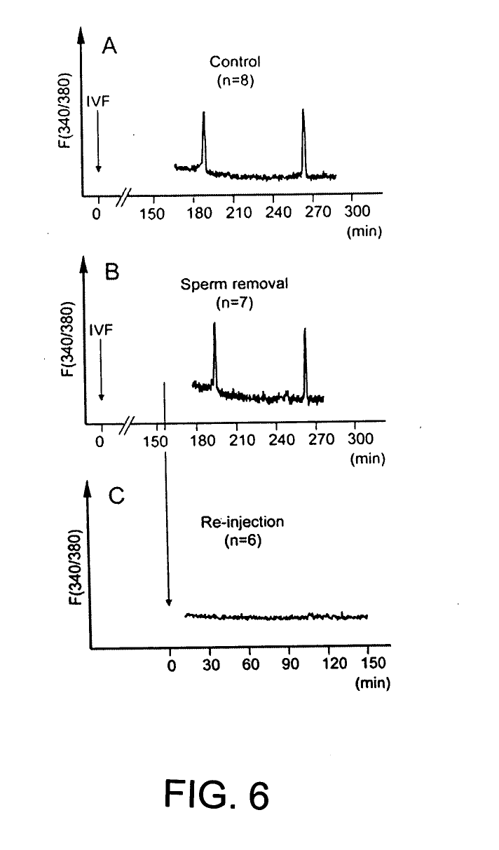

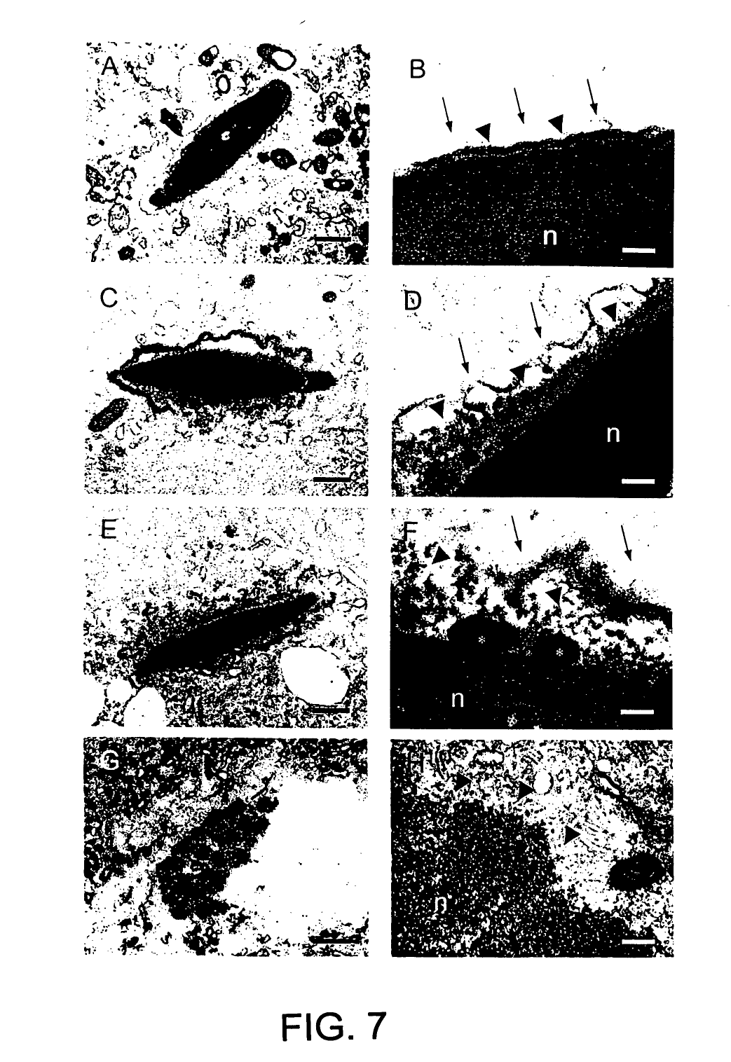

To ascertain whether the temporal release of the factor observed following ICSI was also apparent during natural fertilization, the release of SF was investigated in IVF-generated zygotes. Because IVF is asynchronous and it is difficult to predict the timing of sperm entry, we enucleated eggs at, or immediately after, extrusion of the second polar body, which typically occurs 2.0-2.5 h post-penetration. Removal of sperm did not affect the [Ca²⁺]i responses in the enucleated eggs but, similar to eggs fertilized by ICSI, reinjection of these sperm failed to trigger oscillations in all six examined eggs (FIG. 6A-C).

Whether or not release of SF is specifically promoted by egg factors, or occurs simply by diffusion following dissolution of the sperm membranes, is not known. To test this, sperm heads were first sonicated with Triton X-100 (0.1% Triton for 15 s at 4° C.) followed by thorough washings in IB and incubation in this buffer for 120 min, a time that was sufficient for eggs to deplete the sperm's Ca²⁺ activity, and then injected into MII eggs. Incubation of permeabilized sperm heads in injection buffer did not affect the sperm's Ca²⁺-releasing activity, and the treated sperm consistently induced long-lasting oscillations (n=8; data not shown). Thus, the release of SF does not occur simply by diffusion and may require specific conditions that are readily available in the ooplasm.

Exposure of the Sperm 's PT Coincides with Initiation of [Ca²⁺]i Oscillations

To ascertain the probable location of SF within the sperm head, the morphological changes that occur in the sperm were examined early during fertilization and that accompany the initiation of oscillations. Specifically, the status of the sperm's PT was examined by using TEM at 15, 30, 60, and 120 min post-ICSI. When observed 15 min after ICSI, the sperm heads were highly condensed and both plasma and acrosomal membranes were present, as was the PT (FIGS. 7A and B). In contrast, the plasma membrane was partially lost and the acrosomal membranes appeared partially disintegrated 30 and 60 min after ICSI (FIGS. 7C and D, E and F). Importantly, at these time points, the PT material was exposed, and in some cases partially lost, and the digested material was visible in the surrounding cytoplasm. At 120 min post-fertilization, the sperm heads were decondensed and devoid of acrosomal membranes and PT (FIGS. 7G and H). Therefore, our results in conjunction with those of others (Kimura et al., 1998, Perry et al., 1999) suggest an association between exposure/loss of the PT and initiation of oscillations at fertilization.

Complete Disassembly of the PT is not Required for Initiation of[Ca²⁺]i Oscillations

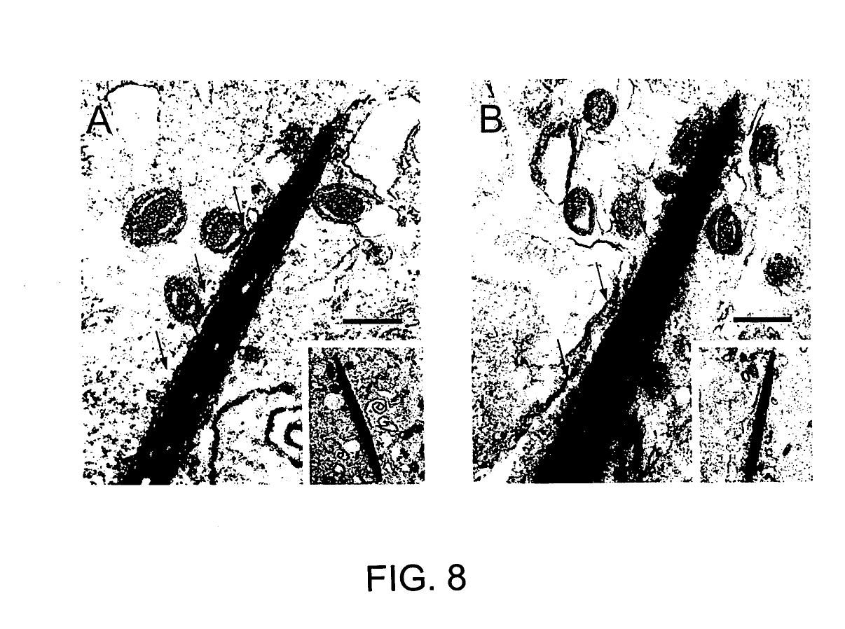

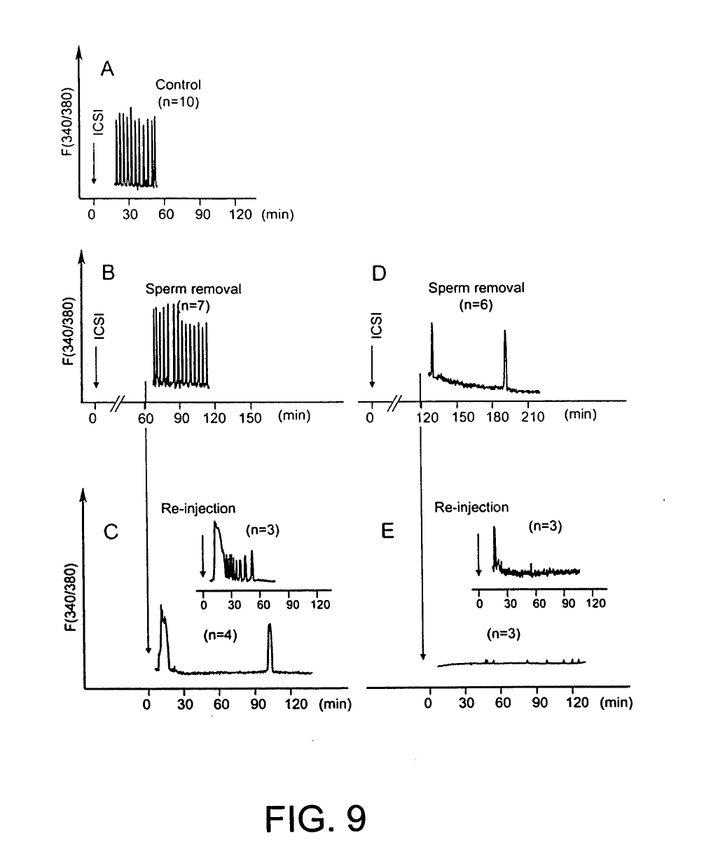

Although SF may be associated with the PT, whether or not the release of the factor relies on the disassembly of the PT is not known and could not be ascertained in the previous experiment. Nonetheless, to address this question, bull sperm heads were injected into mouse eggs; in mouse eggs, bull sperm remain highly condensed and fail to form a PN (unpublished observations). In keeping with these observations, 30 and 120 min post-ICSI, bull sperm retained the PT (FIGS. 8A and B), but even under these conditions they triggered persistent [Ca²⁺]i oscillations (n=5; FIG. 9A). Moreover, bull sperm heads withdrawn 60 (n=7) and 120 (n=6) min after ICSI, which maintained nearly intact PTs, showed negligible [Ca²⁺]i oscillation activity when injected into MII eggs (FIG. 9B-E). Together, these findings suggest that SF is not a structural component of the PT, and its release does not depend on the disassembly of the PT.

Reassociation of SF with the Male PN

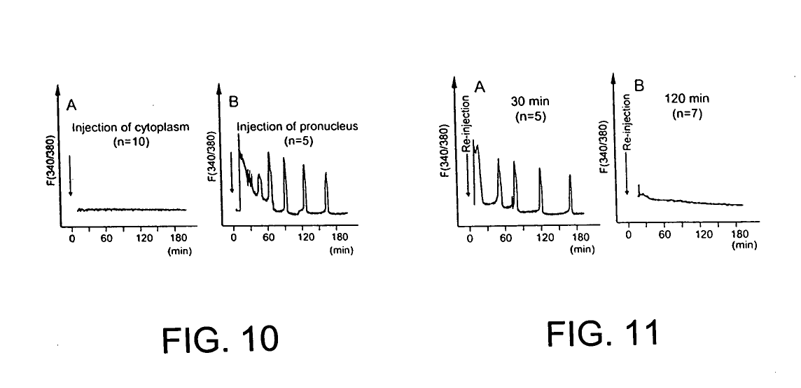

Previous results show that SF is fully released from the sperm into the ooplasm within 60 min of entry. Notably, earlier studies, in what appears to be contradictory results, have demonstrated that SF is associated with PN structures (Kono et al., 1995). Therefore, if both findings are correct, it could be predicted that the factor reassociates with PN structures after spending several hours dispersed in the ooplasm. To test this possibility, male PN removed from zygotes 5-7 h post-ICSI were reinjected into MII eggs. Injection of male pronuclei triggered [Ca²⁺]i oscillations in six of eight examined eggs, confirming the presumed association of SF with nuclear structures, whereas injection of comparable volumes of ooplasm failed to elicit [Ca²⁺]i oscillations (FIG. 10).

The Release of SF Does Not Depend on the Stage of the Cell Cycle

Fertilization in mammals takes place in eggs arrested at the MII stage, a stage that is characterized by high levels of MPF and MAPK activity. To determine whether or not these kinases are important in the release of SF, mouse sperm heads were injected into fertilized PN stage zygotes, which contain inherently low levels of these kinases. After 30 and 120 min in the ooplasm, the sperm heads were recovered and injected into MII eggs and the resulting [Ca²⁺]i responses were monitored. Reinjection of sperm heads withdrawn after 30 min of residence in PN stage zygotes initiated [Ca²⁺]i oscillations (n=5), although with increased intervals, which was similar to the effect induced by incubation in MII eggs (FIG. 11A); sperm recovered 120 min after ICSI were devoid of activity (n=7; FIG. 11B).

The above specification, examples and data provide a complete description of the manufacture and use of the composition of the invention. Since many embodiments of the invention can be made without departing from the spirit and scope of the invention, the invention resides in the claims hereinafter appended.

1. A method for cell activation, the method comprising:
(a) introducing a sperm into a mammalian cell;
(b) culturing the cell for a time sufficient for cell activation; and
(c) removing the sperm from the cell.

2. The method of claim 1, wherein the sperm comprises an intact sperm.

3. The method of claim 1, wherein the sperm comprises a sperm head.

4. The method of claim 1, wherein the sperm comprises a mammalian sperm.

5. The method of claim 4, wherein the mammalian sperm comprises a sperm of a mammal selected from the group consisting of a human, a primate, a bovine, a porcine, an ovine, an equine, a feline, a canine, a caprine, a rabbit, and a rodent.

6. The method of claim 5, wherein the mammalian sperm comprises a human sperm.

7. The method of claim 1, wherein the sperm is heterologous to said mammalian cell to be activated.

8. The method of claim 1, wherein the cell comprises a mammalian cell of a mammal selected from the group consisting of a human, a primate, a bovine, a porcine, an ovine, an equine, a feline, a canine, a caprine, a rabbit, and a rodent.

9. The method of claim 8, wherein the cell comprises a human cell.

10. The method of claim 1, wherein the embryo is selected from the group consisting of a naturally occurring embryo, an embryo produced by in vitro fertilization, a nuclear transfer embryo, and a uniparental embryo.

11. The method of claim 1, wherein the cell has been treated, either before or after introducing the sperm, to remove or inactivate its endogenous nucleus.

12. The method of claim 1, wherein the culturing is performed in vitro or in vivo.

13. The method of claim 12, wherein the culturing is performed in vitro and further comprises incubating the injected cell in a medium containing calcium

14. The method of claim 1, further comprising the step of injecting the cell with one or more agents that enhance divalent cation release in the cell.

15. The method of claim 14, wherein the agent comprises a calcium ionophore, a protein kinase inhibitor, a phosphatase, or a combination thereof.

16. The method of claim 1, wherein the cell comprises an oocyte or an embryo, and further comprising culturing the activated cell to undergo embryonic development.

17. The method of claim 1, wherein the sperm is removed from the cell 15, 30, or 60 minutes following injection.

18. An embryo produced by the method of claim 16, wherein the embryo comprises 1 cell to about 400 cells.

19. The embryo of claim 18, further comprising a blastocyst.

20. A non-human embryo produced by the method of claim 16.

21. The embryo of claim 20, further comprising a blastocyst.

22. The method of claim 16, wherein said embryo is non-human, and further comprising implanting the non-human embryo into a female surrogate.

23. The method of claim 22, wherein said non-human embryo is allowed to develop into a viable, non-human offspring.

24. A non-human offspring produced by the method of claim 23.

25. The method of claim 1, wherein the cell is an oocyte or an embryo, and further comprising induction of persistent calcium oscillations within the oocyte or embryo.

26. An activated mammalian cell produced by the method of claim 1.

27. A method for nuclear transfer cloning comprising:
(a) introducing a mammalian donor cell, or a nucleus derived therefrom into a mammalian enucleated oocyte of the same species as the donor cell or donor cell nucleus, to thereby form a nuclear transfer unit; and
(b) activating the oocyte, wherein the activating comprises:
(i) injecting a sperm into the oocyte;
(ii) culturing the oocyte for a time sufficient for activation; and
(iii) removing the sperm from the oocyte.

28. The method of claim 27, wherein the activating is performed prior to, simultaneous with, or subsequent to the introducing a mammalian donor cell.

29. The method of claim 27, wherein the sperm is heterologous to the oocyte.

30. The method of claim 27, further comprising culturing the nuclear transfer unit to produce an embryo.

31. An embryo produced by the method of claim 30, wherein the embryo comprises 1 cell to about 400 cells.

32. The embryo of claim 31, further comprising a blastocyst.

33. A non-human embryo produced by the method of claim 30.

34. The embryo of claim 33, further comprising a blastocyst.

35. The method of claim 30, wherein the embryo comprises a non-human embryo, and further comprising implanting the non-human embryo into a female surrogate

36. The method of claim 35, wherein said non-human embryo is allowed to develop into a viable, non-human offspring.

37. The non-human offspring produced by the method of claim 36.

38. The method of claim 27, wherein the sperm is removed from the oocyte 15, 30 or 60 minutes following implantation.

39. A method for nuclear transfer cloning comprising:
(a) activating a mammalian oocyte, the activating comprising:
(i) injecting a sperm into the oocyte;
(ii) culturing the oocyte for a time sufficient for activation; and
(iii) removing the sperm from the oocyte;
(b) enucleating the oocyte; and
(c) introducing into the activated enucleated oocyte a mammalian donor cell, or a nucleus derived therefrom, wherein the donor cell is of the same species as the oocyte, to thereby form a nuclear transfer unit.

40. The method of claim 39, wherein the activating is performed prior to, simultaneous with, or subsequent to the enucleating.

41. The method of claim 39, further comprising culturing the nuclear transfer unit to produce an embryo.

42. An embryo produced by the method of claim 41, wherein the embryo comprises from about 1 cell to about 400 cells.

43. The embryo of claim 42, further comprising a blastocyst.

44. A non-human embryo produced by the method of claim 41.

45. The embryo of claim 44, further comprising a blastocyst.

46. The method of claim 41, wherein the embryo comprises a non-human embryo, and further comprising implanting the non-human embryo into a female surrogate

47. The method of claim 46, wherein said non-human embryo is allowed to develop into a viable, non-human offspring.

48. The non-human offspring produced by the method of claim 47.

49. The method of claim 39, wherein the sperm is removed 15, 30, or 60 following implantation.

50. A method for in vitro fertilization, the method comprising:
(a) contacting a mammalian oocyte with a plurality of sperm, whereby the oocyte is fertilized; and
(b) activating the oocyte, wherein the activating comprises:
(i) injecting a sperm into the oocyte;
(ii) culturing the oocyte for a time sufficient for activation; and
(iii) removing the sperm from the oocyte.

51. The method of claim 50, wherein the contacting is performed prior to, simultaneous with, or subsequent to the activating.

52. An embryo produced by the method of claim 50.

53. The method of claim 50, further comprising implanting the embryo into a female surrogate.

54. The method of claim 53, wherein said embryo is allowed to develop into a viable offspring.

55. A non-human offspring produced by the method of claim 54.

56. The method of claim 50, wherein the sperm is removed from the oocyte 15, 30 or 60 min following implantation.

57. The method of claim 16, wherein said embryo is human, and further comprising implanting the human embryo into a female surrogate.

58. The method of claim 22, wherein said human embryo is allowed to develop into a viable human offspring.

59. The method of claim 30, wherein the embryo comprises a human embryo, and further comprising implanting the human embryo into a female surrogate.

60. The method of claim 59, wherein said human embryo is allowed to develop into a viable human offspring.
